(19) United States
(12) Patent Application Publication
(10) Pub. No.: US 2024/0257953 A1
(43) Pub. Date: Aug. 1, 2024

(54) DISTRIBUTION OF SURPLUS PRODUCTS USING ARTIFICIAL INTELLIGENCE

(71) Applicant: INTERNATIONAL BUSINESS MACHINES CORPORATION, Armonk, NY (US)

(72) Inventors: Sarah Diane Green, Chandler, AZ (US); Ira L. Allen, Dallas, TX (US); Stan Kevin Daley, Española, NM (US); Michael Jack Martine, Chapel Hill, NC (US)

(21) Appl. No.: 18/160,625

(22) Filed: Jan. 27, 2023

(51) Int. Cl. G16H 40/20; G06N 20/00

(52) U.S. Cl. CPC G16H 40/20; G06N 20/00

(57) ABSTRACT

At least one surplus distribution model can be trained by machine learning implemented using historical data. The surplus distribution model can be configured to process current data and, based on processing the current data, output first data indicating recommendations for distribution of surplus products. The current data can be processed, using the at least one surplus distribution model. Based on the processing of the current data, the first data indicating the recommendations for distribution of the surplus products can be output. The recommendations for the distribution of the surplus products optimize the distribution of the surplus products based on demand for the surplus products.

### BACKGROUND

The present invention relates to machine learning.

Machine learning is a branch of artificial intelligence (AI) and computer science. Machine learning involves the use and development of computer systems that are able to learn and adapt without following explicit instructions. Aspects of machine learning utilize algorithms and statistical models to analyze and draw inferences from patterns in data.

### SUMMARY

A method can include training, by machine learning implemented using a processor and using historical data, at least one surplus distribution model configured to process current data and, based on processing the current data, output first data indicating recommendations for distribution of surplus products. The method also can include processing, using the at least one surplus distribution model, the current data and, based on the processing of the current data, outputting the first data indicating the recommendations for distribution of the surplus products, the recommendations for the distribution of the surplus products optimizing the distribution of the surplus products based on demand for the surplus products.

A system includes a processor programmed to initiate executable operations. The executable operations can include training, by machine learning implemented using historical data, at least one surplus distribution model configured to process current data and, based on processing the current data, output first data indicating recommendations for distribution of surplus products. The executable operations also can include processing, using the at least one surplus distribution model, the current data and, based on the processing of the current data, outputting the first data indicating the recommendations for distribution of the surplus products, the recommendations for the distribution of the surplus products optimizing the distribution of the surplus products based on demand for the surplus products.

A computer program product includes a computer readable storage medium having program code stored thereon. The program code is executable by a data processing system to initiate operations. The operations can include training, by machine learning implemented using historical data, at least one surplus distribution model configured to process current data and, based on processing the current data, output first data indicating recommendations for distribution of surplus products. The operations also can include processing, using the at least one surplus distribution model, the current data and, based on the processing of the current data, outputting the first data indicating the recommendations for distribution of the surplus products, the recommendations for the distribution of the surplus products optimizing the distribution of the surplus products based on demand for the surplus products.

This Summary section is provided merely to introduce certain concepts and not to identify any key or essential features of the claimed subject matter. Other features of the inventive arrangements will be apparent from the accompanying drawings and from the following detailed description.

### BRIEF DESCRIPTION OF THE DRAWINGS

FIG. 1 is a block diagram illustrating an example of a network data processing system according to an embodiment of the present invention.

FIG. 2 depicts an artificial intelligence supply chain manager according to an embodiment of the present invention.

FIG. 3 depicts a block diagram illustrating machine training of a surplus distribution model according to an embodiment of the present invention.

FIGS. 4A and 4B, together, are a block diagram depicting implementation of a surplus product distribution system according to an embodiment of the present invention.

FIG. 5 is a flowchart depicting a method of optimizing distribution of surplus products.

### DETAILED DESCRIPTION

This disclosure relates to machine learning, as well as optimization and application of machine learning artificial intelligence (AI) models. In this regard, the arrangements described herein are directed to computer technology, and provide an improvement to computer technology. Specifically, the present arrangements improve machine learning as applicable to optimizing delivery of surplus products to those in need of the surplus products. Such process involves machine learning optimized AI models that analyze an impact of surplus product distribution, and optimize surplus product distribution for the greatest overall benefit of those needing the surplus products.

Supply chains generate surplus products and, generally, do not efficiently direct surplus to entities that can consume it. For example, food is habitually wasted at all stages of the supply chain, and waste is built into the processes used by farms, food producers, retail stores and restaurants. Nonetheless, food insufficiency and food insecurity remain a problem for some people. Moreover, disasters create intense widespread food insecurity and a myriad of challenges in the supply chain. Thus, reducing waste and optimizing the distribution of surplus products, such as food, is beneficial for those people who need the surplus products. There is myriad of obstacles in doing so, however, for example lack of interest, logistics, costs, and funding. The arrangements described herein help to streamline the surplus product supply chain and ensure that surplus that would otherwise be wasted can be distributed to entities that can consume it. Food is one just example of a type of surplus product to which the present arrangements are applicable. Other examples of surplus products to which the present arrangements are applicable include surplus medicine, consumables, technology, and apparel, though the present arrangements are not limited to these examples.

Currently, much of the work to distribute surplus products in a supply chain is performed by volunteers in ad-hoc networks. Although the networks use social media to coordinate volunteers, the process itself of organizing volunteers and finding donations is a manual effort. A limitation of existing solutions is the narrow scope of solutions that can track surplus products, for example via blockchain, is that the solutions almost entirely focus on safety. The arrangements described herein not only ensure on safety, but also distribute surplus items to those in need using an intelligent workflow. In this regard, existing solutions do not address the entire supply chain with an intelligent workflow. Known solutions focus on the supply side only, and in the case of food, they emphasize food safety, rather than addressing the demand side.

An artificial intelligence supply chain manager (AISCM) described herein implements AI and machine learning to manage the surplus commodity supply chain, catching and preventing waste, and then optimize distribution to entities that can consume perishable and non-perishable commodities. AISCM also can implement use of blockchains, the Internet of Things (IoT), edge computing, and smart contracts. The present arrangements are not limited to distribution of commodities, however; distribution of surplus designer goods can also be managed by AISCM in accordance with the arrangements described herein.

On the supply side, AISCM can employ multiple technologies to prevent surplus/waste before it occurs, and can direct surplus products that would otherwise be wasted to those who need it. This can include using the IoT sensors/devices, as well as signals from cameras, logistics systems, etc. to monitor the surplus products, and using AI and machine learning to optimize distribution of the surplus products to have greatest beneficial impact. By way of example, cameras or other sensors can be used to monitor freshness of surplus products.

On the demand side, AISCM can use AI and machine learning analyze and optimize factors such as transportation required, available equipment for safe handling and storage, nutrition requirements of local populations, optimal recipes to minimize waste, ability of entities (e.g., soup kitchens) to utilize the ingredients or prepared dishes, and prevailing laws that affect how the food can be distributed. In a medical scenario, AISCM can optimize distribution based on the ability to store the surplus products and qualified medical personnel to assist in disbursal. In the case of other types of products, for example, optimization can include considering weather when deciding where to divert winter coats, and considering joblessness or other social factors when diverting cosmetics and apparel that could be worn on job interviews. In a catastrophe scenario, AISCM can manage distribution of aid in accordance with the arrangements described herein to achieve greatest benefit from the aid.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as

AISCM 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

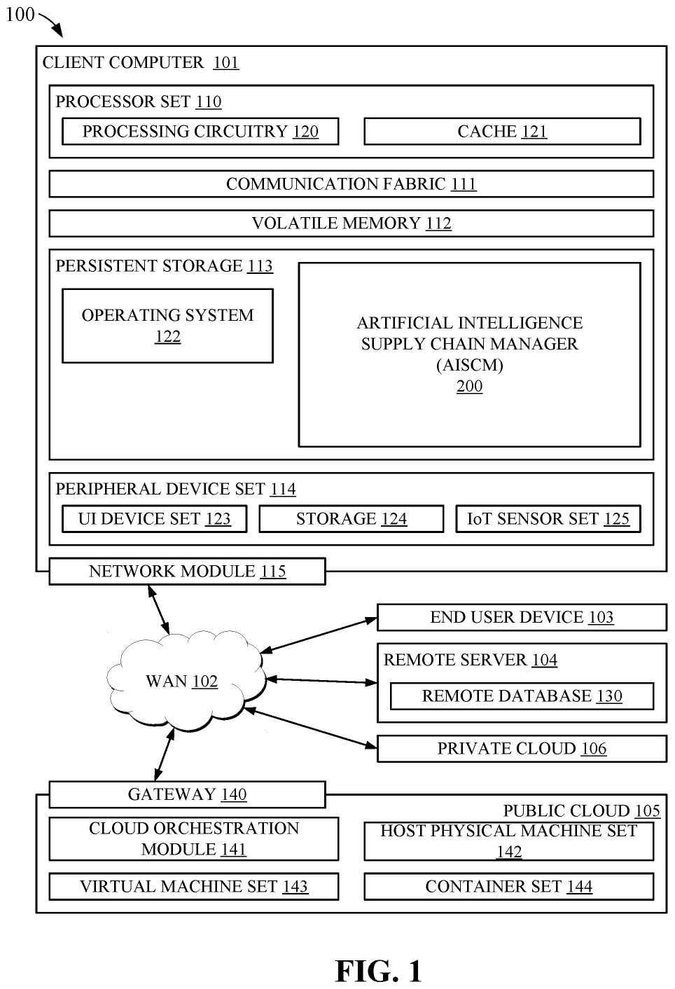

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

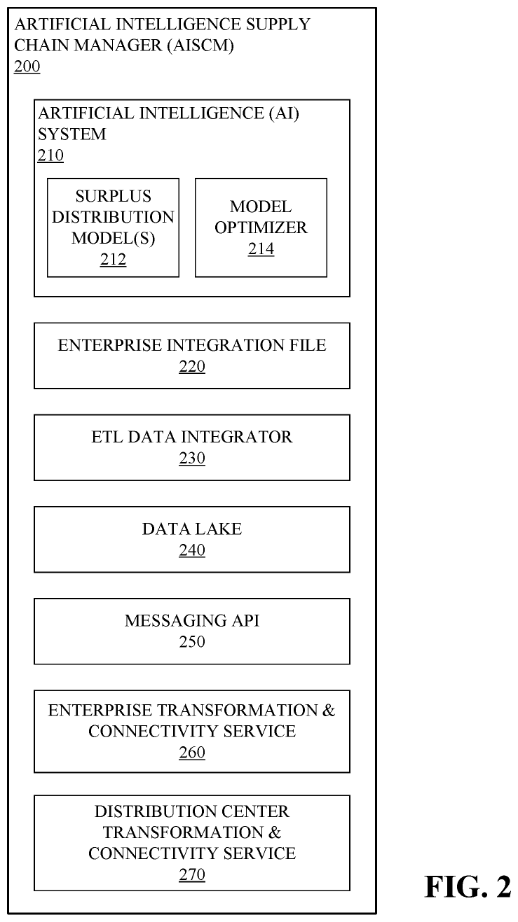

FIG. 2 depicts an artificial intelligence supply chain manager (AISCM) 200 according to an embodiment of the present invention. AISCM 200 can include an artificial intelligence (AI) system 210. AI system 210 can include one or more surplus distribution models 212 and a model optimizer 214, which are further described herein.

AISCM 200 also can include an enterprise integration file 220. Enterprise integration file 220 can include data describing how data from enterprises is to be integrated by AISCM 200. In illustration, AISCM 200 also can include an extract, transform and load (ETL) data integrator 230. ETL data integrator 230 can integrate data in enterprise integration file 220 and data received from various other data sources, which will be described herein, into a data lake 240 according to enterprise integration file 220. In this regard, ETL data integrator 230 can combine data from multiple data sources into a data lake 240. In one or more arrangements, data lake 240 can be a single, consistent data store that is loaded into AISCM 200 for performing various operations described herein, including training of surplus distribution model(s) 212 and for input to surplus distribution model(s) 212 to optimize distribution of surplus products. In this regard, data received from various data sources may be formatted differently, may contain extraneous data not needed by AISCM 200, etc. ETL data integrator 230 can cleans and organize data according to the specific needs of AISCM 200, for example using a series of data cleansing rules. Accordingly, ETL data integrator 230 can improve data quality and establish consistency of data used by AISCM 200.

AISCM 200 also can include a messaging application programming interface (API) 250. AISCM 200 can utilize messaging API 250 to generate messages and communicate the messages to various enterprises and/or people. The generated messages can be, for example, electronic messages (e.g., email messages and/or text messages), telephone messages generated by AISCM 200 using text-to-speech synthesis, data packets communicated to other data processing systems, or any other type of messages that may be used to communicate information. AISCM 200 also can utilize messaging API 250 to receive messages. The generated messages can be, for example, electronic messages (e.g., email messages and/or text messages), telephone messages, data packets received from other data processing systems, or any other type of messages that may be used to communicate information. In the case of telephone messages, in one or more arrangements AISCM 200 can implement speech recognition to convert the messages to text. In one or more arrangements, AISCM 200 can be commutatively linked to one or more cloud native message hubs (not shown). API 250 can send and receive messages via the cloud native message hub(s).

AISCM 200 also can include an enterprise transformation and connectivity service (ETCS) 260. ETCS 260 can be configured to interface with a variety of enterprise systems, for example product assortment and planning systems, core retail systems, forecast and demand planning systems, transport management systems and warehouse management systems, as will be described.

AISCM 200 also can include distribution center transformation and connectivity service (DCTCS) 270. DCTCS 270 can be configured to interface with distribution center systems, for example automated handling systems and distribution center devices, as will be described. Examples of distribution center devices include, but are not limited to, workstations, personal computers, tablet computers, mobile phones, etc. DCTCS 270 also can be configured to receive sensor data from distribution center sensors/devices, for example Internet of Things (IoT) sensors/devices.

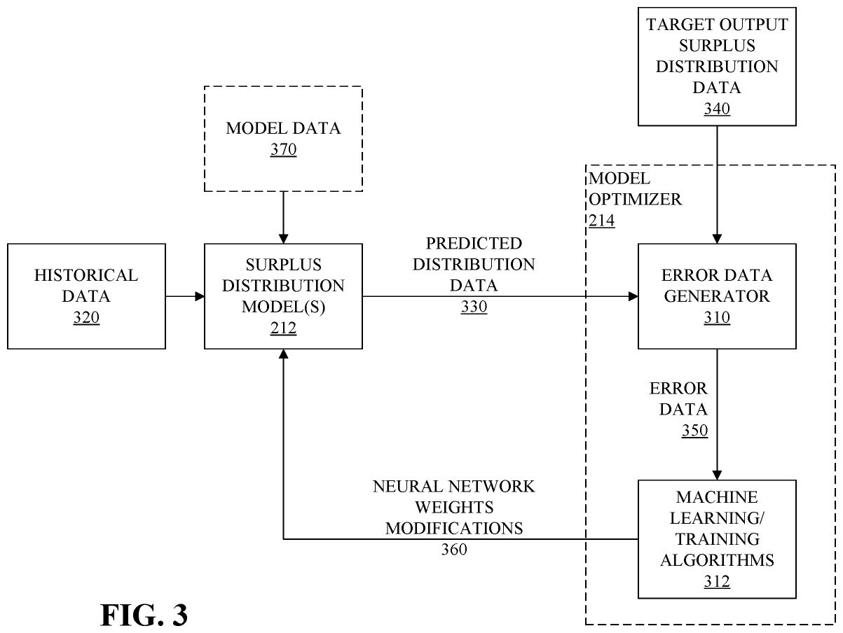

FIG. 3 depicts a block diagram illustrating machine training of a surplus distribution model 212 according to an embodiment of the present invention. Surplus distribution model 212 can be trained by model optimizer 214.

Model optimizer 214 can include error data generator 310 and machine learning/training algorithms 312 configured to train AI models, such as surplus distribution models 212 described herein. Surplus distribution models 212 can be trained to estimate impact, on both observable and unobservable surplus product distribution effects, of known and/or unknown decision variables and other observable explanatory variables over multiple future time periods.

Surplus distribution model(s) 212 can receive, as input training data for machine learning, historical data 320. Historical data 320 can include, for example, observed distributions of surplus products. Historical data 320 can include both structured and unstructured data. The structured data can include, for example, historical decisions (e.g., distribution decisions made for various surplus products and types of surplus products) and contexts pertaining to such decisions. Example of such contexts can include, but are not limited to, demand for surplus products, including demand for specific products, in specific geographic areas, surplus product quantities delivered, surplus product expiration dates, medical information (e.g., medical ailments including, but not limited to, vitamin deficiencies) prevalent in geographic areas to which surplus products are distributed, means of transportation used to distribute the surplus products, weather conditions, as a time series, for geographic areas where the surplus products are distributed, vehicle traffic conditions on various roadways, as a time series, for geographic areas where the surplus products are distributed, data pertaining to size of surplus product storage/distribution centers, data pertaining equipment (e.g., refrigerators) at surplus product storage/distribution centers, etc. Other examples of the structured data include, but are not limited to, data pertaining to disease outbreaks, catastrophes, demographic information, etc. The structured data also can include results corresponding to the historical decisions (e.g., consumption statistics on distributed surplus products) and other observable explanatory variables. The historical decisions and results may be partially observed, influencers including decision variables and other explanatory variables, which are recorded in a time series manner, etc.

Unstructured data can include results of performing natural language processing (NPL) on media content (e.g., news, social media, event information, scientific papers, medical papers, social media content, etc.) which may have an impact on surplus product distribution decisions over time. In illustration, if a medical paper discusses a particular medical ailment (e.g., a vitamin deficiency) prevalent in a particular geographic area, the unstructured data can include data indicating that particular medical ailment in that particular geographic area. In one or more arrangements, AISCM 200 can include a NLP module that receives and processes media content to generate unstructured data. In one or more arrangements, AISCM 200 can utilize another system or service to receive and process the media content to generate unstructured data, and AISCM 200 can receive the unstructured data from that system or service.

Unstructured data also can include results of processing images (e.g., satellite imagery, maps, weather maps, photographs, etc.), processing videos, processing audio recordings, processing metadata included in image files, processing metadata included in video files, processing metadata included in audio files, and so on. In illustration, image recognition can be performed on images and/or videos to determine geographic areas, places, things and/or people in the images and/or videos. Speech-to-text and NLP processing can be performed on videos and/or audio recordings to determine meaning of audio content. Metadata extraction can be performed on image files, video files, audio files, etc. to extract metadata from such files as unstructured data. In one or more arrangements, AISCM 200 can include one or more modules that receive images, videos, audio recordings, etc. and perform image processing, audio processing, NLP, metadata extraction, etc. on such content. In one or more arrangements, AISCM 200 can utilize another system or service to that receive images, videos, audio recordings, etc. and perform image processing, audio processing, NLP, metadata extraction, etc. on such content.

By way of example, elements depicted in videos and/or still images in and/or around a building can indicate a specific location of the building (e.g., buildings address, etc.) and/or a geographic area where the building is located. Geospatial data indicated in maps also can be used into indicate a specific location and/or geographic area where a building or other structure is located.

A geographic area can be a continent, a nation, a state, a county, a province, a city, a village, a neighborhood, a portion of a continent, a portion of a nation, a portion of a state, a portion of a county, a portion of a province, a portion of a city, a portion of a village, a portion of a neighborhood, etc.

Historical data 320 can be granular in nature. For example, historical data 320 can include data for various periods (e.g., times of day, days of the week, weekends, weeks, months, holidays, etc.), and include such data for various types of surplus products and/or distribution services. Surplus distribution model 212 can be trained to select explanatory variables from historical data 320, which contain decision information, and estimate optimal allocation of surplus products. Surplus distribution model 212 also can be trained to formulate explanatory variables based on historical data 320, estimate surplus product consumption, etc.

Surplus distribution model 212 can process historical data 320 using surplus product demand algorithms and generate predicted distribution data 330 based on such processing. Error data generator 310 can compare predicted distribution data 330 to target output surplus distribution data 340, and generate error data 350 indicating differences between predicted distribution data 330 and to target output surplus distribution data 340. Machine learning/training algorithms 312 can process the error data 350 and, based on such processing, generate neural network weights modifications 360. Machine learning/training algorithms 312 can modify coefficients of the surplus distribution model's artificial neural network (ANN) using the neural network weights modifications 360, in a process known as machine learning (or training). In addition to the historical data 320, model data 370 generated by the trained surplus distribution model 212 (e.g., as predicted distribution data 330) can be provided as input to the surplus distribution model 212 during training of the surplus distribution model 212.

The machine learning process can iterate until an objective is satisfied. Such objective can be, for example, generation of error data 350 having one or more values below one or more threshold values, the objective can be to iterate the machine learning process a threshold number of times, or the objective can be to iterate the machine learning process a threshold duration of time. Moreover, the machine learning process can be iterated in response to additional historical data 320 being acquired. Accordingly, forecasting results corresponding to optimal decisions for each period can be iteratively refreshed, and organizational objectives can be maximized over multiple future time periods.

In one or more arrangements, surplus distribution model(s) 212 can be trained as a time series model (e.g., an autoregresive distributed lag (ARDL) model, an autoregressive integrated moving average (ARIMA) model, or any other AI model suitable for training as a time series model), based on observed demand and various other factors. The time series mode can be implemented to process current data and, based on processing the current data, predict total demand for a surplus product, and strategies for optimizing distribution of the surplus products based on the demand, over a future time series. In illustration, surplus distribution model(s) 212 can predict the demand (e.g., need) for various surplus products in various geographic areas, and predict strategies to best meet that demand, while ensuring that those who are most in need of the surplus products receive distribution of the surplus products.

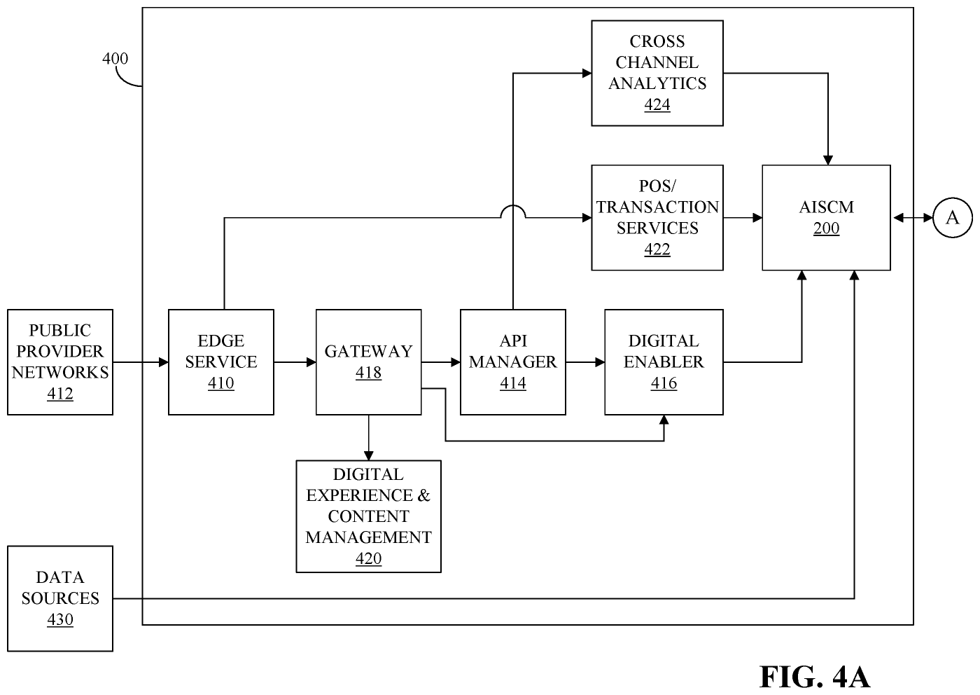

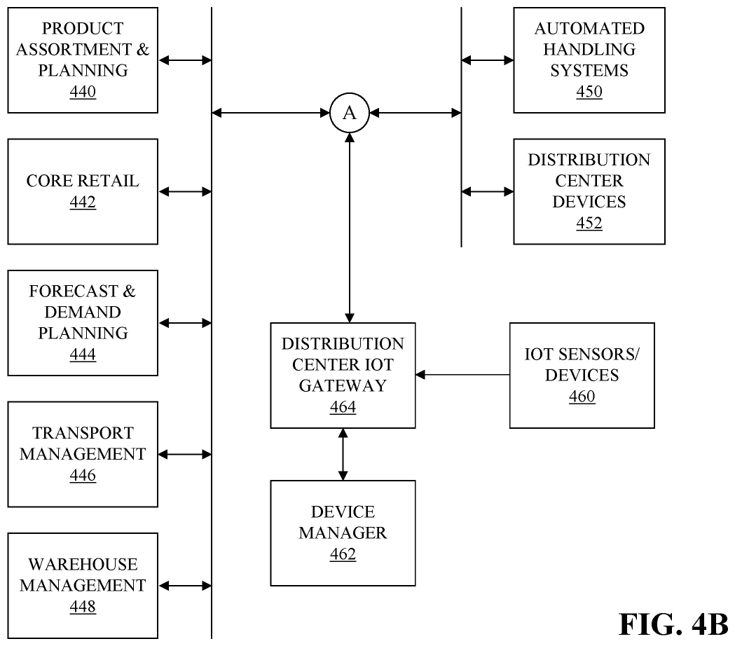

FIGS. 4A and 4B together are a block diagram depicting implementation of a surplus product distribution system (hereinafter “system”) 400 according to an embodiment of the present invention. System 400 can include AISCM 200.

System 400 further can include an edge service 410 via which members of public provider networks 412 can communicate with system 400. Public provider networks 412 can include, for example, shoppers, customers, application users in agencies, vendors, suppliers, wholesalers, freight consolidators, etc. Edge service 410 can be communicatively linked to other components of system 400, for example an API manager 414 and digital enabler 416, via a gateway 418. Gateway 418 can provide ingress control to system 400 and a service mesh. Gateway 418 also can be used to output, from system 400, digital experience and content management 420 data. Digital experience and content management 420 can provide user experiences for consuming the output of AISCM 200, such as recommendations output by AISCM 200. Digital experience can be presented as an application (e.g., desktop application, web application, mobile application, etc.) configured to present content to users. Digital experience can include a user friendly front end taking into account culturally appropriate natural language generation and understanding. Digital experience can include modules that present content using mixed reality (e.g., augmented reality, virtual reality, etc.). Further, digital experience can include exposed APIs for consumption of content by other systems. such as enterprise resource planning systems, point of sale systems that track inventory, and so on.

Edge service 410 also can provide access by public provider networks 412 to point of sale (POS)/transaction services 422. For example, via edge service 410 a thrift store can access POS/transaction services 422 to communicate data indicating surplus items distributed, surplus items needed, etc. In this regard, demand can be captures from endpoints that provide feeding demand signals system 400. Those demand signals could include, for example, indications of surplus product demand (e.g., “more like this,” “less like this,” “no more winter coats, as it's now springtime, but come back in the fall,” etc.) that provide data AISCM 200 can process to further optimize supply/demand machine learning algorithms implemented by surplus distribution models 212, including temporal aspects. Accordingly, surplus distribution models 212 can evolve and improve over time, thus improving recommendations made by AISCM 200 over time.

API manager 414 can manage various APIs utilized by system 400 including, but not limited to, API 250, APIs used by digital enabler 416, and APIs used for cross channel analytics 424. APIs used by digital enabler 416 can include, for example, various APIs for credit/rewards management, commerce/catalog management, data searches, order fulfilment/management, enterprise marketing management, sources of surplus product relationships/loyalty management, partner and ecosystem services, etc. Cross channel analytics 424 can provide advanced analytics of data across multiple surplus product distribution channels. In illustration, cross channel analytics 424 can include analyses of data output by ETCS 260 and DCTCS 270, as well as analyses of channel data output by other systems/modules.

AISCM 200 can receive, as input, data generated by digital enabler 416, POS/transaction services 422 and cross channel analytics 424. AISCM 200 (e.g., ETCS 260) also can receive data from various other data sources, including data sources 430. Examples of such data sources 430 include, but are not limited to, sources that provide information pertaining charities, government agencies, food and/or medication information, medical papers, other medical information, scientific papers, other scientific information, weather information, satellite imagery, safety information, vendor/supplier/wholesaler/supplier information, transportation provider information, etc. Structured and/or unstructured data can be provided by data sources 430.

AISCM 200 (e.g., via ETCS 260) can communicate with a plurality of enterprise systems including, but not limited to, product assortment and planning systems 440, core retail systems 442, forecast and demand planning systems 444, transport management systems 446 and warehouse management systems 448. Product assortment and planning systems 440 can include, for example, product development data and category assortment data for a variety of products. Core retail systems 442 can include, for example, product pricing data, contractual data, inventory management data, sourcing management data, supply chain transparency data and a supplier collaboration hub. Forecast and demand planning systems 444 can include, for example, product forecast data, store replenishment data and warehouse replenishment data. Transport management systems 446 can include, for example, route planning data and transport network optimization data. Warehouse management systems 448 can include, for example, material handling data and stock handling data.

AISCM 200 (e.g., via DCTCS 270) can communicate with a plurality of distribution center systems/devices including, but not limited to, automated handling systems 450 and distribution center devices 452. Automated handling systems 450 can include, for example, automated material handling systems and automated stock handling systems. Distribution center devices 452 can include, for example, client devices.

AISCM 200 (e.g., via DCTCS 270) also can receive sensor data from distribution center IoT sensors/devices 460. The sensor data can include, for example, data indicating stock levels of products, data indicating how long products have been in stock, data indicating movement of products in distribution centers, and so on. Further, AISCM 200 (e.g., via DCTCS 270) can interface with distribution center device managers 462, for example those that manages IoT devices. AISCM 200 can interface with IoT sensors/devices 406 and device managers 462 via a distribution center IoT gateway 464.

AISCM 200 can process data received from digital enabler 416, POS/transaction services 422, channel analytics 424, data sources 430, systems 440-448, systems 450-452, IoT sensors/devices 460 and/or device managers 462 using enterprise, and integrate such data into data lake 240 using enterprise integration file 220 and ETL data integrator 230. AISCM 200 can access the data from data lake 240 and process the data. Based on such processing, AISCM 200 can determine surplus products available for distribution, and determine how and where to distribute the surplus products in order to optimize their distribution. In this regard, AISCM 200 can provide the data as input data to one or more surplus distribution models 212 that have been trained using machine learning, and output data generated by the surplus distribution models 212.

AISCM 200 can utilize the data generated by the surplus distribution models 212 in a variety of ways in order to optimize surplus product distribution. For example, via ETCS 260, AISCM 200 can place orders for surplus products directly with core retail systems 442. Via DCTCS 270, AISCM 200 can place orders for surplus products directly with distribution centers, and provide control instructions for the automated handling systems 450 to prepare surplus products for shipment. Via DCTCS 270, AISCM 200 also can interface with device managers 462 to provide control instructions to IoT sensors/devices 460. Further, via ETCS 260, AISCM 200 can interface with transport management systems 446 to schedule pickup of surplus items from retail locations and/or distribution centers and schedule delivery of surplus items to locations where the surplus items can be provided to those who need the surplus items. When selecting delivery locations and scheduling transportation, AISCM 200 can account for travel time (e.g., based on weather conditions, traffic conditions and/or distances between pickup locations and delivery locations) and determinations of whether the travel times will result in possible expiration of perishable items (e.g., food and medicine). AISCM 200 can dynamically expand and constrict the delivery radius based on the shelf stability of the surplus products. For example, fruit/medicine that is about to become overripe requires faster transportation, refrigeration, etc., and may be directed to closer recipients, while nonperishable goods, such as those that do not require temperature controlled transit or storage, can be transported to a more distant recipient.

In illustration, AISCM 200 can identify surplus products, which can include both perishable and non-perishable products (e.g., food, clothing, medicine, etc.). AISCM 200 can recognize the potential surplus could be time/season based, and automated via sensors, such as cameras, which detect when a product is past its prime (tomato plants at the end of the growing season, bananas on the grocery store shelf, or even fashion such as a particular clothing item that no longer commands the full retail price). AISCM 200 can compare the anticipated value of the discounted price to the market (gleaned from published and certified sources, (e.g., tax authorities and third-party sources) against the potential financial consequences, and may also consider the local state of the economy, weather, etc., to determine the optimal repositioning of the goods.

In this regard, AISCM 200 can make optimal distribution determinations based on very complex sets of parameters, including source based parameters and demand based parameters. AISCM 200 can examine data and input the data into surplus distribution models 212, which can be multi-dimensional models, to match surplus products to recipients who need those products.

AISCM 200 can prioritize recipient organizations, using AI, considering factors such as capacity, transportation, location, aggregated demographics, government information, catastrophe data (e.g., satellite imaging after a natural disaster) to get near real-time validation, third-party data such as catastrophe maps (e.g., from an insurance company) and/or current demographic circumstances. AISCM 200 can validate recipient entities using exogenous data from trusted and reliable sources (e.g., Charity Navigator). Accordingly, surplus products can be aggregated in a manner that is tuned to the ability of organizations to warehouse, distribute and/or otherwise use the surplus goods. Moreover, AISCM 200 can perform AI processing on data in real time, thus accounting for conditions that currently exist, and giving less weight, or no weight, to conditions that no longer exist.

By way of example, when a natural disaster occurs (flood, earthquake, fire, hurricane, tornado, etc.) and/or there is a large relocation of people/livestock, there is an immediate need in the supply chain to react to those changing conditions. AISCM 200 can anticipate the humanitarian and animal needs based on geo-spatial data (e.g., satellite imagery), and place a demand in the system for food, water, shelter, medicine, etc. for distribution by non-governmental organizations and governmental agencies that have the capacity to distribute the products to populations that need them. Accordingly, AISCM 200 can anticipate and avoid gluts of products which, if they are perishable in nature, would result in much waste.

With AISCM 200 outputting recommendations using AI and based on both historical data (e.g., historical supply and demand data) and current data (e.g., current supply and demand data), recommendations output AISCM 200 can be free of biases that sometimes influence human decisions. In illustration, when choosing surplus products and choosing where to distribute surplus products, some people may have bias toward particular types of surplus products, certain surplus product suppliers, certain geographic areas where surplus products are to be distributed, certain means of transportation, etc. Such biases can result in sub-optimal correspondence between available surplus products and to whom those surplus products are distributed. AISCM 200 can avoid such biases, and provide recommendations that optimally match available surplus products to those who need the surplus products. Such equitable distribution of surplus products can serve to manage, and potentially improve, the reputations of various organizations (e.g., charities, enterprises, etc.) participating in surplus product distribution. Accordingly, using AISCM 200, organizations can manage reputational risk. In this regard, AISCM 200 can serve to manage reputational risk for various organizations participating in the distribution of surplus products.

Moreover, based on historical and current demand data, AISCM 200 can determine demand for combinations of surplus products in different geographic areas, and output recommendations including surplus product combinations selected for geographic areas where those combinations are in demand. In illustration, in a first geographic area a combination of product A with product B may be high in demand, while in a second geographic area a combination of product A with product C may be high in demand. Thus, AISCM 200 can recommend distribution of products A and B to the first geographic area, and recommend distribution of products A and C to the second geographic area.

AISCM 200 can identify the needs of groups, as well as communities that serve individuals in need. In the realm of food delivery to those in need, AISCM 200 goes beyond merely assessing food insecurity/insufficiency, but also can determine needs for specific nutrients. For example, a particular geographic area might have a high percentage (e.g., a percentage above a threshold level) of individuals suffering from a particular medical deficiency, which AISCM 200 can determine by processing data from data sources 430 (e.g., news articles, medical papers, scientific papers, etc.). The medical deficiency can be, for example, a nutritional deficiency (e.g., a vitamin deficiency). AISCM 200 can help alleviate the medical deficiency by outputting recommendations for delivery to that geographic area of surplus foods, vitamins, medicines, etc. that will alleviate the medical deficiency. By way of example, if a high percentage of individuals are suffering from a vitamin D deficiency, AISCM 200 can recommend delivery to those individuals of surplus food and/or vitamins rich in vitamin D. Thus, beyond optimizing for reaching individuals based on broad categories such as proximity, family size, available transportation, etc., AISCM 200 can optimize surplus product distribution to direct nutrients to those who most need those nutrients.

AISCM 200 can determine surplus foods, surplus vitamins, surplus medicines, etc., that alleviate medical deficiencies by processing data retrieved from data sources 430. For example, AISCM 200 can process medical information and/or nutritional information indicating nutrients provided by various foods, vitamins, medicines, etc. The medical information can include, but is not limited to, medical journal articles, scientific papers, etc. Based on processing the data, AISCM 200 can determine foods, vitamins, medicines, etc. that alleviate medical deficiencies, and determine which of those items are available for distribution as surplus products. AISCM 200 can recommend for delivery to geographic areas the surplus products that will elevate the medical deficiencies in those geographic areas.

AISCM 200 can identify recipient distribution organizations, which may depend on volunteers to reach individuals, and/or they may enable self-service by individuals. AISCM 200 can enable operation at any level, for example at a national level, a state level, a municipal level, or at an individual level. AISCM 200 can change the scale of work and necessity across growing or shrinking groups.

In one or more arrangements, AISCM 200 can serve recipient individuals directly. For example, individuals can register to receive notifications that a store that distributes surplus products not only has items that the individual can buy (e.g., with subsidies), but also may have surplus products that the recipient can obtain at no cost. AISCM 200 can maintain privacy, for example using suitable security protocols, while still reaching individuals. Nonetheless, in one or more arrangements AISCM 200 need not require that individuals register to receive surplus products.

AISCM 200 can consider what surplus products are available and how those products can be combined to create additional value to those in need. For example, AISCM 200 can provide to individuals recipes to make meals using the surplus products provided to those individuals. By way of example, AISCM 200 can specify that the necessary ingredients for a recipe be assembled into a box that is delivered to an individual. In some cases, all ingredients called for by a particular recipe may not be available, but AISCM 200 can access data from various data sources to determine substitutions for unavailable products using available surplus products. For example, assume a recipe is for a cake, and the recipe typically specifies that oil be used in the cake recipe, but the oil is not available to the individual as a surplus product. Also, assume that applesauce may be substituted for the oil to make the cake, and applesauce is available to the individual as a surplus product, AISCM 200 can change the recipe to substitute applesauce for the oil, and recommend that applesauce be included with the surplus items to be delivered to the individual.

By recommending surplus product distribution at an individual level, AISCM 200 can help target recipients maximize their nutritional intake, optimizing what is available and tailoring to their preferences for consumption. In this regard, AISCM 200 also can take into consideration individual medical histories, if provided, and output surplus product recommendations optimized for individuals based on the individuals' needs. For example, AISCM 200 can refrain from recommending surplus products to individual who are allergic to those products. Accordingly, AISCM 200 can generate recommendations with a confidence level that entity can safely consume or otherwise use the surplus products.

Beyond the donor who has the surplus product and the receiving organization or individual, AISCM 200 can determine other entities available to participate in storing and/or distribution of surplus products, or provide other services to needy individuals, and consider those other entities as participants. For example, a restaurant might be able to donate use of a freezer to save surplus food until a recipient organization can use it, or a transportation company can donate or sell its services to transport surplus products. A phone carrier can donate phones that can be used by the end user (individual). The phones can include an application the end user can use to connect to available surplus products at various types of providers (food bank, soup kitchen, obtain directly from store, medical clinic, etc.). In one or more arrangements, AISCM 200 can process data from data sources 430 to determine the other entities available to participate in storing and/or distribution of surplus products, or provide other services to needy individuals. In one or more arrangements, AISCM 200 can receive information (e.g., via electronic messaging or a web site) offering to provide services to help needy individuals. In one or more arrangements, AISCM 200 can communicate electronic messages to various entities to recruit those entities to provide services to help needy individuals.

AISCM 200 can access from various data sources data for smart contracts. AISCM 200 can process data to automatically generate smart contracts that provide certified immutable records, such as receipts and/or accounting records, of surplus product donation transactions, as well as other donation transactions. Each immutable record representing a donation can include, for example, a block generated by AISCM 200 in a blockchain. Immutable records also can be generated using technologies similar to blockchains. Smart contracts can cover donors and receivers, and can be useful for financial planning purposes, as well as protecting the identities of the individual recipients. In one or more arrangements, blocks generated by AISCM 200 in a blockchain can match receipts/accounting records for donations and smart contracts to donors and other entities participating in distribution of surplus products. AISCM 200 also can generate journal entries configured to be automatically entered into accounting systems.

Further, AISCM 200 can access data from various data sources for prevailing laws and regulations in locations being serviced for surplus product deliveries. AISCM 200 can analyze how laws and regulations affects what surplus products can be donated and how the surplus products can be distributed. AISCM 200 can generate surplus product delivery recommendations that comply with those laws and regulations.

In one or more arrangements, laws and regulations accessed by AISCM 200 can include tax laws and tax regulations. AISCM 200 can implement surplus distribution models 212 trained by AISCM 200 to assess and determine financial opportunities available for surplus product donors and other entities participating in the charitable distribution of the surplus products. AISCM 200 can output data indicating results of such assessments and determinations, and communicate such data to the surplus product donors and other entities, for example using messaging API 250, ETCS 260 and/or DCTCS 270. In illustration, entities can be provided limited access to AISCM 200 using client devices (e.g., via an application or web portal), and enter information pertaining to products/services available to be donated. AISCM 200 can assess the products/services, determine financial opportunities available for such donations, and present information indicating such financial opportunities via the client devices. Accordingly, decisions pertaining to charitable contributions can be made with knowledge of financial impacts for those contributions. AISCM 200 can analyze data indicating partnerships that are outside of a products typical supply chain, and not normally handles by organizations that typically serve disadvantaged people. Examples of such partnerships can include partnerships with ride share companies and/or delivery companies that can sell or donate rides to transport surplus products, for example from a restaurant to a soup kitchen. Another example of a partnerships is a partnership with phone carrier that distributes donation phones, which can be distributed to end users who receive donations of food.

In one or more arrangements, AISCM 200 can implement surplus distribution models 212 trained by AISCM 200 to output data pertaining to accountability, auditing and validation. In illustration, AISCM 200 can receive data pertaining to how surplus products were distributed (e.g., to which charities were the surplus products donated), and the demand for those surplus products (e.g., how long did the surplus products sit on store shelves). Based on such data, AISCM 200 can further train surplus distribution models 212, used to determine how surplus products are to be distributed, to increase the effectiveness of the surplus product distribution. In this regard, AISCM 200 can reconcile surplus product donations to consumption patterns of those surplus products and determine efficacy of surplus product distribution. AISCM 200 can, for example, assign efficacy ratings to various organizations participating in the distribution of surplus products, including efficacy ratings on a per product basis and overall efficacy ratings. Based on reconciliating surplus product donations and the efficacy ratings, AISCM 200 can optimize, and thus improve, surplus product distribution. For example, if there is low demand for product A distributed through charity A, but there is a high demand for product A distributed through charity B, AISCM 200 can train surplus distribution models 212 to recommend a greater portion of future distributions of product A go to charity B.

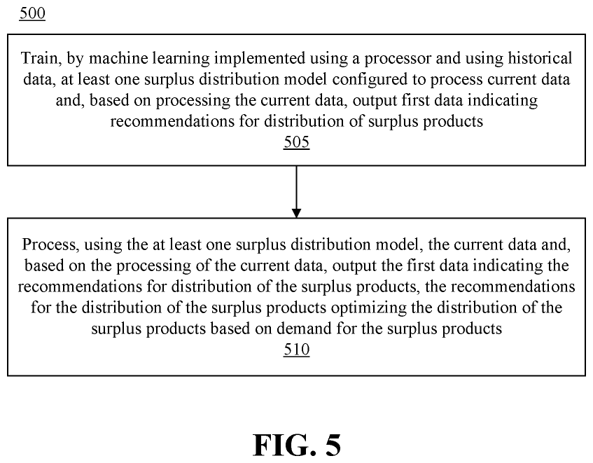

FIG. 5 is a flowchart depicting a method 500 of optimizing distribution of surplus products. The method 500 can be implemented by AISCM 200.

At step 505 AISCM 200 can train, by machine learning implemented using a processor and using historical data, at least one surplus distribution model configured to process current data and, based on processing the current data, output first data indicating recommendations for distribution of surplus products

At step 510 AISCM 200 can process, using the at least one surplus distribution model, the current data and, based on the processing of the current data, output the first data indicating the recommendations for distribution of the surplus products, the recommendations for the distribution of the surplus products optimizing the distribution of the surplus products based on demand for the surplus products

The foregoing description is just an example of embodiments of the invention, and variations and substitutions. While the disclosure concludes with claims defining novel features, it is believed that the various features described herein will be better understood from a consideration of the description in conjunction with the drawings. The process(es), machine(s), manufacture(s) and any variations thereof described within this disclosure are provided for purposes of illustration. Any specific structural and functional details described are not to be interpreted as limiting, but merely as a basis for the claims and as a representative basis for teaching one skilled in the art to variously employ the features described in virtually any appropriately detailed structure. Further, the terms and phrases used within this disclosure are not intended to be limiting, but rather to provide an understandable description of the features described.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “includes,” “including,” “comprises,” and/or “comprising,” when used in this disclosure, specify the presence of stated features, integers, steps, operations, elements, and/or components, but do not preclude the presence or addition of one or more other features, integers, steps, operations, elements, components, and/or groups thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “includes,” “including,” “comprises,” and/or “comprising,” when used in this disclosure, specify the presence of stated features, integers, steps, operations, elements, and/or components, but do not preclude the presence or addition of one or more other features, integers, steps, operations, elements, components, and/or groups thereof.

Reference throughout this disclosure to “one embodiment,” “an embodiment,” “one arrangement,” “an arrangement,” “one aspect,” “an aspect,” or similar language means that a particular feature, structure, or characteristic described in connection with the embodiment is included in at least one embodiment described within this disclosure. Thus, appearances of the phrases “one embodiment,” “an embodiment,” “one arrangement,” “an arrangement,” “one aspect,” “an aspect,” and similar language throughout this disclosure may but do not necessarily, all refer to the same embodiment.

The term “plurality,” as used herein, is defined as two or more than two. The term “another,” as used herein, is defined as at least a second or more. The term “coupled,” as used herein, is defined as connected, whether directly without any intervening elements or indirectly with one or more intervening elements, unless otherwise indicated. Two elements also can be coupled mechanically, electrically, or communicatively linked through a communication channel, pathway, network, or system. The term “and/or” as used herein refers to and encompasses any and all possible combinations of one or more of the associated listed items. It will also be understood that, although the terms first, second, etc. may be used herein to describe various elements, these elements should not be limited by these terms, as these terms are only used to distinguish one element from another unless stated otherwise or the context indicates otherwise.

The term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As defined herein, the term “surplus product” means a product that is donated for charitable distribution.

As defined herein, the term “client device” means a data processing system that requests shared services from a server, and with which a user directly interacts. Examples of a client device include, but are not limited to, a workstation, a desktop computer, a computer terminal, a mobile computer, a laptop computer, a netbook computer, a tablet computer, a smart phone, a personal digital assistant, a smart watch, smart glasses, a gaming device, a set-top box, a smart television and the like. Network infrastructure, such as routers, firewalls, switches, access points and the like, are not client devices as the term “client device” is defined herein.

As defined herein, the term “real time” means a level of processing responsiveness that a user or system senses as sufficiently immediate for a particular process or determination to be made, or that enables the processor to keep up with some external process.

As defined herein, the term “output” means storing in memory elements, writing to display or other peripheral output device, sending or transmitting to another system, exporting, or similar operations.

As defined herein, the term “automatically” means without user intervention.

As defined herein, the term “individual” means a person (i.e., a human being).

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
training, by machine learning implemented using a processor and using historical data, at least one surplus distribution model configured to process current data and, based on processing the current data, output first data indicating recommendations for distribution of surplus products; and
processing, using the at least one surplus distribution model, the current data and, based on the processing of the current data, outputting the first data indicating the recommendations for distribution of the surplus products, the recommendations for the distribution of the surplus products optimizing the distribution of the surplus products based on demand for the surplus products.

2. The method of claim 1, wherein:
the current data indicates a medical deficiency prevalent in a geographic area; and
the recommendations for the distribution of the surplus products comprise a recommendation to distribute the surplus products in a manner that alleviates the medical deficiency.

3. The method of claim 1, wherein:
the current data indicates a vitamin deficiency prevalent in a geographic area; and
the recommendations for the distribution of the surplus products comprise a recommendation to distribute to that geographic area surplus foods or vitamins that alleviate the vitamin deficiency.

4. The method of claim 1, further comprising:
based on the processing of the current data, outputting second data, the second data comprising surplus product recommendations optimized for individuals based on the individuals' needs.

5. The method of claim 1, further comprising:
based on the processing of the current data, outputting second data, the second data comprising a recipe for a meal to be made from the surplus products to be delivered to an individual.

6. The method of claim 5, further comprising:
wherein the recipe substitutes at least one of the surplus products for a product typically used in the recipe but that is not available to the individual as a surplus product.

7. The method of claim 1, further comprising:
generating an immutable record representing a donation, the immutable record comprising a block in a blockchain.

8. A system, comprising:
a processor programmed to initiate executable operations comprising:
training, by machine learning implemented using historical data, at least one surplus distribution model configured to process current data and, based on processing the current data, output first data indicating recommendations for distribution of surplus products; and
processing, using the at least one surplus distribution model, the current data and, based on the processing of the current data, outputting the first data indicating the recommendations for distribution of the surplus products, the recommendations for the distribution of the surplus products optimizing the distribution of the surplus products based on demand for the surplus products.

9. The system of claim 8, wherein:
the current data indicates a medical deficiency prevalent in a geographic area; and
the recommendations for the distribution of the surplus products comprise a recommendation to distribute the surplus products in a manner that alleviates the medical deficiency.

10. The system of claim 8, wherein:
the current data indicates a vitamin deficiency prevalent in a geographic area; and
the recommendations for the distribution of the surplus products comprise a recommendation to distribute to that geographic area surplus foods or vitamins that alleviate the vitamin deficiency.

11. The system of claim 8, the executable operations further comprising:
based on the processing of the current data, outputting second data, the second data comprising surplus product recommendations optimized for individuals based on the individuals' needs.

12. The system of claim 8, the executable operations further comprising:
based on the processing of the current data, outputting second data, the second data comprising a recipe for a meal to be made from the surplus products to be delivered to an individual.

13. The system of claim 12, the executable operations further comprising:
wherein the recipe substitutes at least one of the surplus products for a product typically used in the recipe but that is not available to the individual as a surplus product.

14. The system of claim 8, the executable operations further comprising:
generating an immutable record representing a donation, the immutable record comprising a block in a blockchain.

15. A computer program product, comprising:
one or more computer readable storage mediums having program code stored thereon, the program code stored on the one or more computer readable storage mediums collectively executable by a data processing system to initiate operations including:
training, by machine learning implemented using historical data, at least one surplus distribution model configured to process current data and, based on processing the current data, output first data indicating recommendations for distribution of surplus products; and
processing, using the at least one surplus distribution model, the current data and, based on the processing of the current data, outputting the first data indicating the recommendations for distribution of the surplus products, the recommendations for the distribution of the surplus products optimizing the distribution of the surplus products based on demand for the surplus products.

16. The computer program product of claim 15, wherein:
the current data indicates a medical deficiency prevalent in a geographic area; and
the recommendations for the distribution of the surplus products comprise a recommendation to distribute the surplus products in a manner that alleviates the medical deficiency.

17. The computer program product of claim 15, wherein:
the current data indicates a vitamin deficiency prevalent in a geographic area; and
the recommendations for the distribution of the surplus products comprise a recommendation to distribute to that geographic area surplus foods or vitamins that alleviate the vitamin deficiency.

18. The computer program product of claim 15, wherein the program code is executable by the data processing system to initiate operations further comprising:
based on the processing of the current data, outputting second data, the second data comprising surplus product recommendations optimized for individuals based on the individuals' needs.

19. The computer program product of claim 15, wherein the program code is executable by the data processing system to initiate operations further comprising:
based on the processing of the current data, outputting second data, the second data comprising a recipe for a meal to be made from the surplus products to be delivered to an individual.

20. The computer program product of claim 19, wherein the program code is executable by the data processing system to initiate operations further comprising:
wherein the recipe substitutes at least one of the surplus products for a product typically used in the recipe but that is not available to the individual as a surplus product.
